(19) United States
(12) Patent Application Publication
(10) Pub. No.: US 2013/0041314 A1
(43) Pub. Date: Feb. 14, 2013

(54) Steerable Catheters

(71) Applicant: Travis E. Dillon, Winston-Salem, NC (US)

(72) Inventor: Travis E. Dillon, Winston-Salem, NC (US)

(73) Assignee: Cook Medical Technologies LLC, Bloomington, IN (US)

(21) Appl. No.: 13/566,121

(22) Filed: Aug. 3, 2012

Related U.S. Application Data
(60) Provisional application No. 61/522,441, filed on Aug. 11, 2011.

(51) Int. Cl. A61M 25/092

(52) U.S. Cl. 604/95.04

(57) ABSTRACT

A steerable catheter and a method of steering a catheter are provided. The steerable catheter includes a flexible elongate shaft having a proximal end portion, a distal end portion and a cable lumen extending from the proximal end portion to the distal end portion. The steerable catheter includes a control cable having a first portion and a second portion, the first portion of the control cable fixed in position relative to the shaft and the second portion movable relative to the shaft and at least a portion of the second portion is extendable through at least a portion of the cable lumen and extendable through the opening. The control cable has a first position where the at least the portion of the second portion is axially extendable away from a wall of the shaft and a second position for delivery of the catheter.

### RELATED APPLICATIONS

This application claims the benefit of U.S. Provisional Application No. 61/522,441, filed Aug. 11, 2011, which is incorporated by reference herein in its entirety.

### BACKGROUND

The present invention relates to steerable catheters having precise movement and methods of their use.

Steerable catheters known in the art typically have one or more steering cables that extend within one or more lumens in the catheter along the length of the catheter and are anchored at the distal tip of the catheter. The distal tip of the catheter typically has a vertebrate or soft material to facilitate bending of the distal tip. The cables are pulled alternatively to cause bending of the distal tip in a direction that tension is applied. The resulting deflection of the distal tip of the catheter helps the physician to direct the catheter into a particular lumen of the patient.

However, these types of steerable catheters are very expensive and time consuming to manufacture. In addition, if only slight movements of the tip of the catheter are required, the complex and expensive catheter construction may not be necessary.

What is needed is a steerable catheter that is simple and inexpensive to manufacture. A steerable catheter that allows the user to make slight movements in multiple directions and also allows for a pivoting of the distal tip of the catheter is also needed.

### BRIEF SUMMARY

In one aspect, steerable catheter is provided. The steerable catheter includes a flexible elongate shaft having a proximal end portion, a distal end portion and a cable lumen extending from the proximal end portion to the distal end portion. The cable lumen includes an opening at the distal end portion. The steerable catheter also includes a control cable having a first portion and a second portion, the first portion of the control cable fixed in position relative to the shaft and the second portion movable relative to the shaft and at least a portion of the second portion is extendable through at least a portion of the cable lumen and extendable through the opening. The control cable has a first position where the at least the portion of the second portion is axially extendable away from a wall of the shaft and a second position for delivery of the catheter. A handle operably connected to the control cable for moving the control cable from the second position to the first position.

In another aspect, a steerable catheter is provided. The steerable catheter includes a flexible elongate shaft having a proximal end portion, a distal end portion and a plurality of cable lumens extending from the proximal end portion to the distal end portion, each of the plurality of cable lumens includes an opening at the distal end portion. The steerable catheter also includes a plurality of control cables, each of the plurality of control cables having a first portion and a second portion, the first portion fixed in position relative to the shaft and the second portion movable relative to the shaft. Each of the second portions has at least a portion thereof extendable through one the plurality of cable lumens and out through the opening and axially away from a wall of the shaft in a first position. A handle is operably connected to the plurality of control cables to move one or more of the control cables from a second position to the first position.

In yet another aspect, a method of steering a catheter is provided. The method includes moving a second portion of a first control cable from a second cable position to a first cable position and a first portion of the first control cable remains fixed in position in relation to a shaft of the catheter. The method further includes extending at least a portion of the second portion through an opening in the shaft and axially away from a wall of the shaft and contacting a surface with the portion of the second portion so that a distal portion of the shaft is moved from a first shaft position to a second shaft position.

### BRIEF DESCRIPTION OF THE DRAWINGS

FIG. 1 is a partial perspective view of a steerable catheter in a first position in accordance with an embodiment of the present invention;

FIG. 2 is a partial perspective view of the steerable catheter shown in FIG. 1 in a second position;

FIG. 3 is a partial perspective view of the steerable catheter shown in FIG. 1 with the drive cables removed;

FIG. 4 is a side view of a steerable catheter in accordance with an embodiment of the present invention;

FIG. 5 is a partial perspective view of a steerable catheter illustrating steering of the catheter in accordance with an embodiment of the present invention;

FIG. 6 is a partial perspective view of a steerable catheter illustrating steering of the catheter in accordance with an embodiment of the present invention;

FIG. 7 is a perspective view of a steerable catheter in accordance with an embodiment of the present invention;

FIG. 8 is a perspective view of a portion of the handle of the steerable catheter shown in FIG. 7;

FIG. 9 is a perspective view of a distal portion of a steerable catheter in accordance with an embodiment of the present invention;

FIG. 10 is a perspective view of the distal portion of the steerable catheter shown in FIG. 9 in a second position; and

FIG. 11 is a perspective view of a distal portion of a steerable catheter in accordance with an embodiment of the present invention.

### DETAILED DESCRIPTION

The invention is described with reference to the drawings in which like elements are referred to by like numerals. The relationship and functioning of the various elements of this invention are better understood by the following detailed description. However, the embodiments of this invention as described below are by way of example only, and the invention is not limited to the embodiments illustrated in the drawings. It should also be understood that the drawings are not to scale and in certain instances details have been omitted, which are not necessary for an understanding of the present invention, such as conventional details of fabrication and assembly.

As used in the specification, the terms proximal and distal should be understood as being in the terms of a physician using the steerable catheter. Hence the term distal means the portion of the steerable catheter which is farthest from the physician and the term proximal means the portion of the steerable catheter which is nearest to the physician.

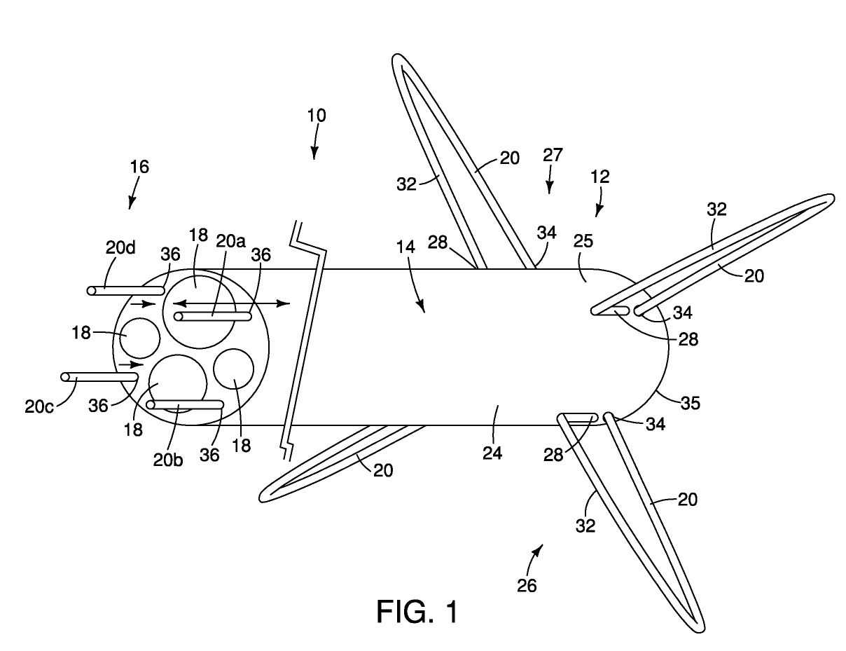

FIG. 1 illustrates a perspective view of a steerable catheter 10 according to an embodiment of the invention. A distal portion 12 of the steerable catheter 10 is shown in FIG. 1. The catheter 10 includes an elongate shaft 14 extending from the distal portion 12 to a proximal portion 16. The elongate shaft 14 includes one or more lumens 18 extending at least partially therethrough. The lumen 18 may be used for devices such as laser and EHL probes, biopsy forceps, baskets, stents and the like. The catheter 10 may also include an image sensor, a light source, irrigation capabilities, aspiration capabilities and the like. The steerable catheter 10 further includes a plurality of control cables 20 extending through at least a portion of the elongate shaft 14. One or more of the plurality of control cables 20 extend outward from an outer surface 24 of the shaft 14 at the distal portion 12 in a first position 26 as shown in FIG. 1. A distal end 28 of each control cable 20 may be secured relative to the shaft 14 with a remaining portion 32 of the control cable 20 movably positionable relative to the shaft 14. The distal portion 12 of the shaft 14 may include a plurality of openings 34 in a wall 25 through which the plurality of control cables 20 may movably extend. As shown in FIG. 1, the openings 34 may be positioned proximal to a distal end 35 of the shaft 14. The plurality of control cables 20 may extend through a plurality of cable lumens 36 to the plurality of openings 34 and out through the openings 34. Each cable 20 may have its own lumen 36 and opening 34. Each cable 20 may be independently moveable and extendable to different distances away from the shaft 14 to position the shaft 14 as described in more detail below. Each cable 20 may extend away from the shaft 14 in a curvilinear shape, such as arcuate or bowed. Each openings 34 may be positioned apart from the distal end 28 of the cable 20 so that the cable 20 forms the curvilinear shape before the cable 20 enters the opening 34.

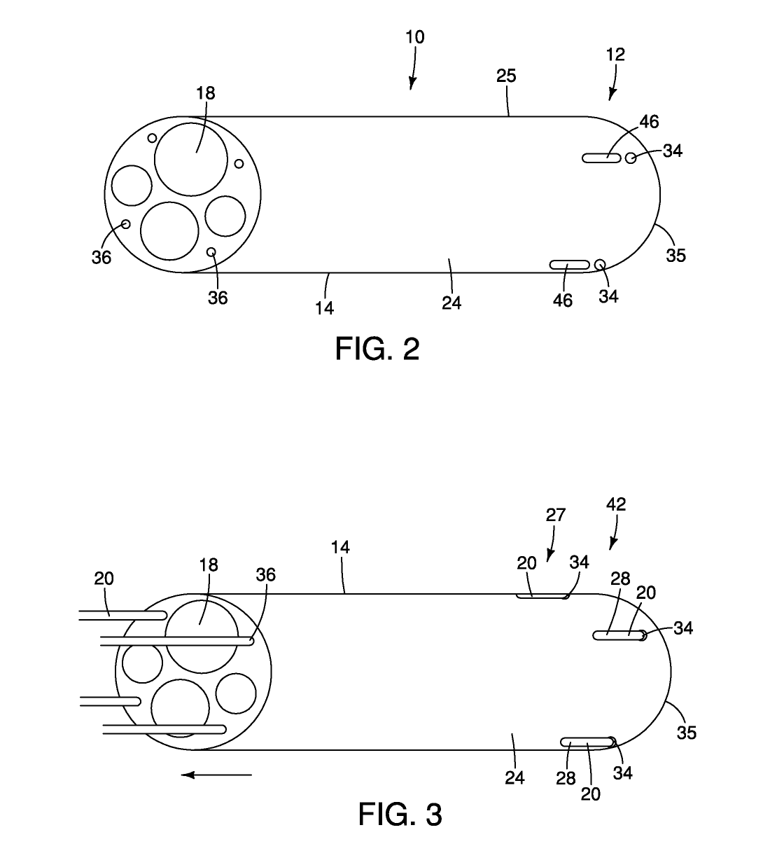

FIG. 2 illustrates the distal portion 12 of the steerable catheter 10 with the control cables 20 in a second position 42. In the second position 42, the control cables 20 are pulled proximally so that the remaining portion 32 of each control cable is withdrawn into the control lumens 36 and do not axially extend away from the shaft 14. The distal end 28 of each control cable 20 remains fixed in position relative to the shaft 14. The second position 42 may be used for delivery of the steerable catheter 10 to a treatment position within the patient's lumen or for repositioning the steerable catheter 10. In the second position 42, the distal end 28 of each control cable 20 may be positioned against the outer surface 24 of the shaft 14. In some embodiments, the distal end 28 of each control cable 20 may be positioned at least partially within a recess 46 formed in the outer surface 24 of the shaft 14 as shown in FIG. 3. FIG. 3 illustrates an exemplary steerable catheter 10 with the control cables 20 removed to illustrate the lumens 36, the openings 34 and the optional recesses 46.

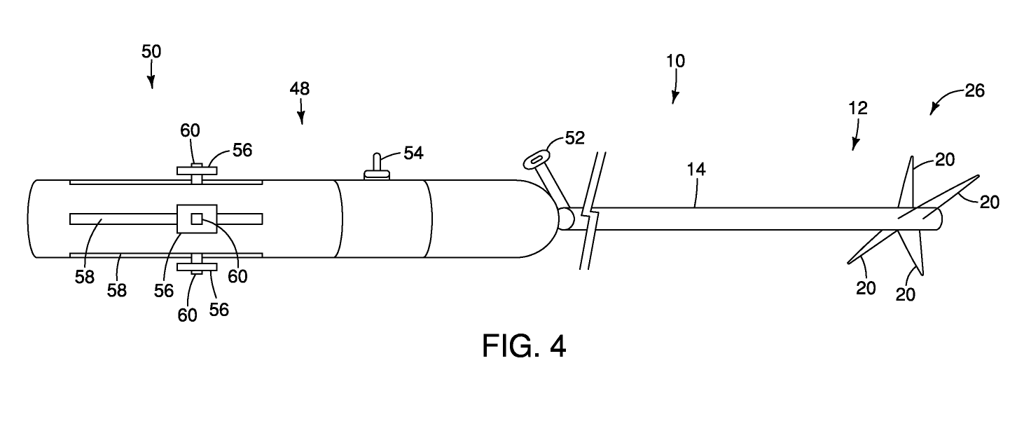

FIG. 4 illustrates an embodiment the steerable catheter 10. The distal portion 12 of the shaft 14 shows the control cables 20 extended axially away from the shaft 14. An exemplary handle 48 is shown at a proximal portion 50 of the catheter 10. The handle 48 may be any kind of handle known to one skilled in the art and may include one or more fluid ports 52 such as a Touhy-Borst adaptor and an electrical connector 54 if the catheter 10 is intended to include an electrified portion such as a sphincterotome. The handle 48 shown in FIG. 4 also includes a plurality of actuators 56 for movably positioning the control cables 20, each actuator 56 may control one cable 20. The exemplary handle 48 illustrates actuators 56 that are longitudinally slidable to control the extension and retraction of the operably attached cable 20. The actuators 56 may be movable within slots 58 formed in the handle 48 so that the operator can grip the handle 48 and move the actuators 56 simultaneously. Each actuator 56 may include a locking mechanism 60 to releasably secure each control cable 20 in position, for example during delivery or once the distal portion 12 is positioned at the treatment site within the patient's lumen. Each actuator 56 may be independently movable so that each control cable 20 may be extended and retracted independently to control the movement of a distal end portion 27 of the catheter 10.

As shown in FIGS. 1 and 4, some embodiments of the steerable catheter 10 may include four control cables 20. In other embodiments, the steerable catheter may include 2, 3 or more control cables 20 that are independently controllable or may be controlled in pairs or other multiples. Table 1 below illustrates exemplary movements that are possible for the embodiments of the steerable catheter 10 including four control cables 20. The control cables 20 have been identified as 20a, 20b, 20c and 20d for illustrative purposes. With additional control cables 20 more directions and combinations are possible and with fewer control cables 20, less directions and combinations are possible. In some embodiments, the control cables 20 may be positioned around the circumference of the shaft 14. The control cables 20 may be equally spaced apart or non-equally spaced. For example, when four control cables 20 are included with the steerable catheter 10, the control cables 20 may be spaced about 90° apart from each other. If two control cables 20 are included, the control cables may be spaced about 180° apart from each other. Other spacing of the control cables 20 is also possible.

TABLE 1

| Wire # | 20a | 20b | 20c | 20d | Tip Direction |
|---|---|---|---|---|---|
| Combination 1 | Push | Push | Pull | Pull | Left |
| Combination 2 | Pull | Pull | Push | Push | Right |
| Combination 3 | Push | Pull | Pull | Push | Down |
| Combination 4 | Pull | Push | Push | Pull | Up |

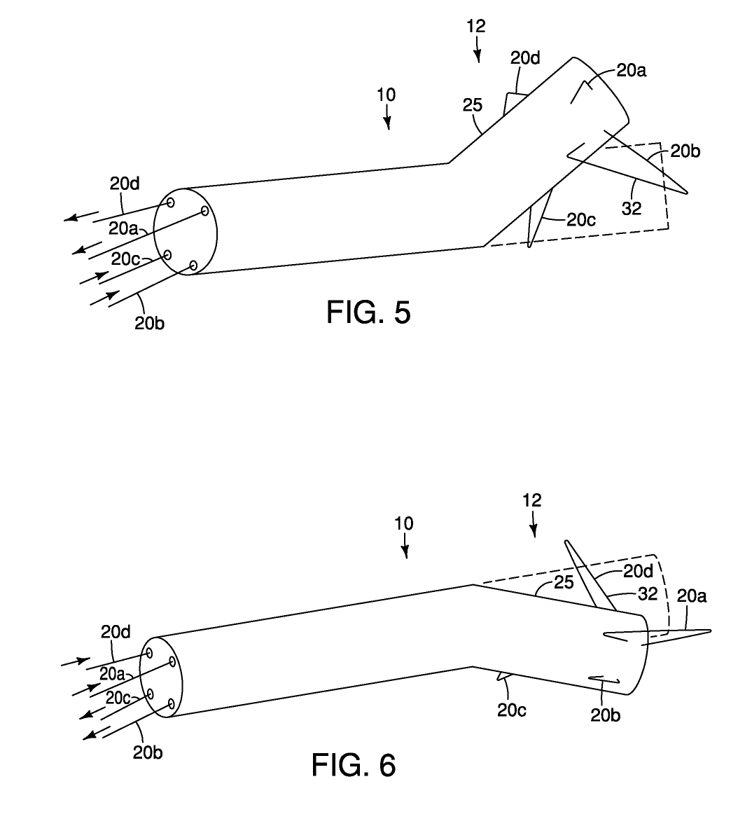

In some embodiments, the amount of movement of the distal portion 12 corresponds to the longitudinal distance that the actuator 56 is pushed distally or pulled proximally to move the cable 20. For example, the shorter the longitudinal distance, the smaller the movement. The small movements of one or more of the control cables 20 allow the operator to precisely control the movement of the distal end portion 27 of the steerable catheter 10. The amount of axial extension of each control cable 20 away from the wall 25 controls the distance that the distal end portion 27 is moved away from the wall of the patient's lumen. To move the distal end portion 27 closer to the wall of the patient's lumen, the amount of axial extension of the control cable 20 is decreased, for example by proximally withdrawing the control cable 20. Exemplary movements of the four control cables 20a-20d relative to each other are illustrated in FIGS. 5 and 6, showing movement of the steerable catheter 10 up or down, respectively. The range of movement shown in FIGS. 5 and 6 may be greater or smaller than that shown.

In some embodiments, all of the control cables 20 may be extended axially away from the shaft 14 to help position the distal end portion 27 of the steerable catheter 10 away from the wall of the patient's lumen. This positioning of the distal end portion 27 away from the wall helps the operator obtain a better view through a lens of a viewing port so that the wall of the patient's lumen does not interfere with the view through the port. The control cables 20 may be used to generally centrally position the distal end portion 27 within the lumen or if not centrally positioned, to hold the distal end portion 27 away from the wall of the patient's lumen.

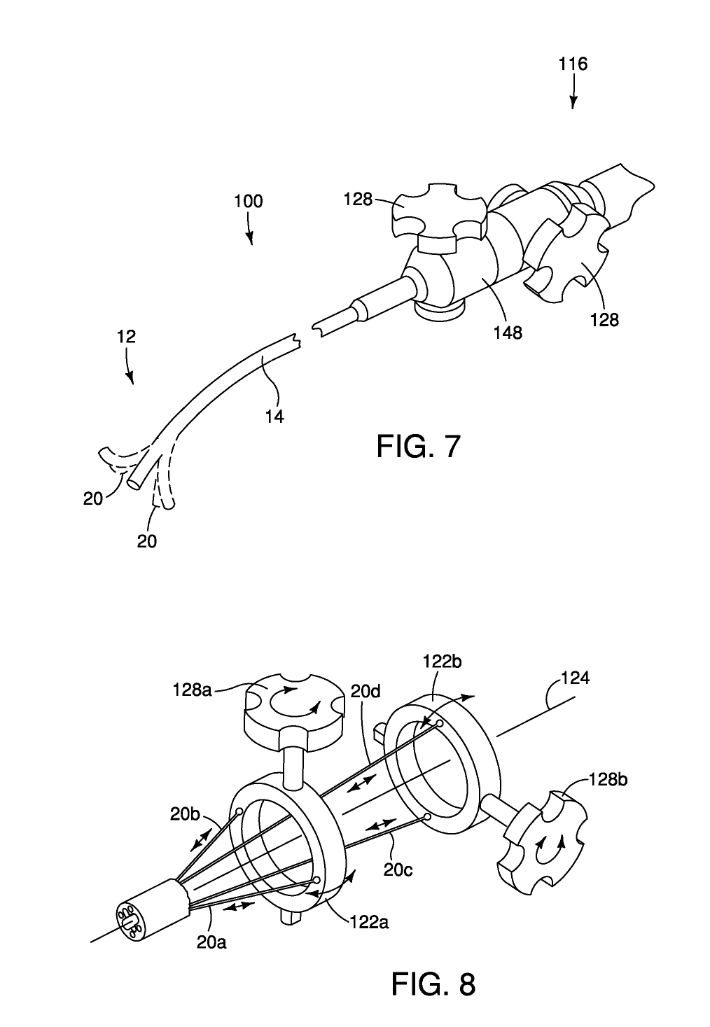

FIGS. 7 and 8 illustrate an alternative embodiment for a handle 148 to control the distal portion 12 of a steerable catheter 100. The steerable catheter 100 is similar to the steerable catheter 10 described above and only the differences in the handle 148 are described. The distal portion 12 of the steerable catheter 100 shown the FIG. 7 illustrates possible positions for the distal portion 12 controlled by the handle 148 moving the control cables 20. The handle 148 may include one or more actuators in shown as ring members 122 rotatable about a longitudinal axis 124. Each ring member 122 may be rotated by a knob 128 to control the movement of the control wires 20 connected to each ring member 122. For example, as shown in FIG. 8, control wires 20a and 20b may be connected to the ring member 122a that is controlled by the knob 128a. When the knob 128a is rotated in a first direction, control wire 20a may be extended distally and control wire 20b may be retracted proximally to control the length of the remaining portion 32 of each control wire that extends away from the wall 25 of the shaft 12 (see FIGS. 5 and 6 illustrating the positions of the control wires at the distal portion 12). Rotating the knob 128a in a second direction allows the control wire 20a to be retracted proximally and the control wire 20b to be extended distally. Similarly, when the knob 128b is rotated in a first direction, control wire 20c may be extended distally and control wire 20d may be retracted proximally to control the length of the remaining portion 32 of each control wire that extends away from the wall 25 of the shaft 12. Rotating the knob 128b in the second direction reverses the extension and retraction of the control wires 20c and 20d. As shown in FIG. 8, the control wires 20c, 20d extend through the ring member 122a and are not controlled by the rotation of the ring member 122a. In some embodiments, the control wires 20c, 20d may be connected to both the ring members 122a, 122b for controlling the extension and retraction of the control wires 20c and 20d (not shown). Additional methods for controlling extension and retraction of the control wires 20 to control the movement of the distal end portion 27 of the catheter 10, 100 may also be used.

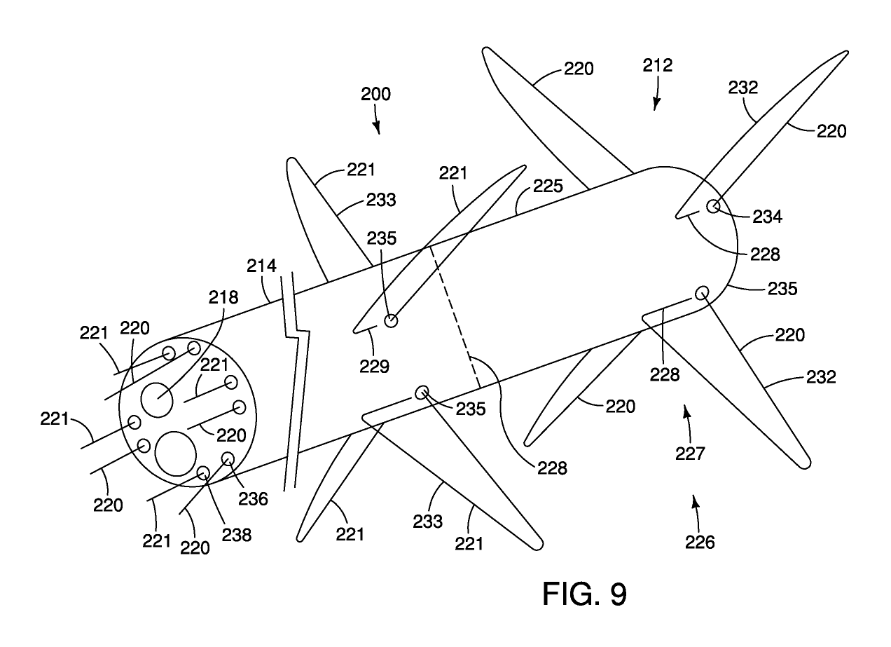

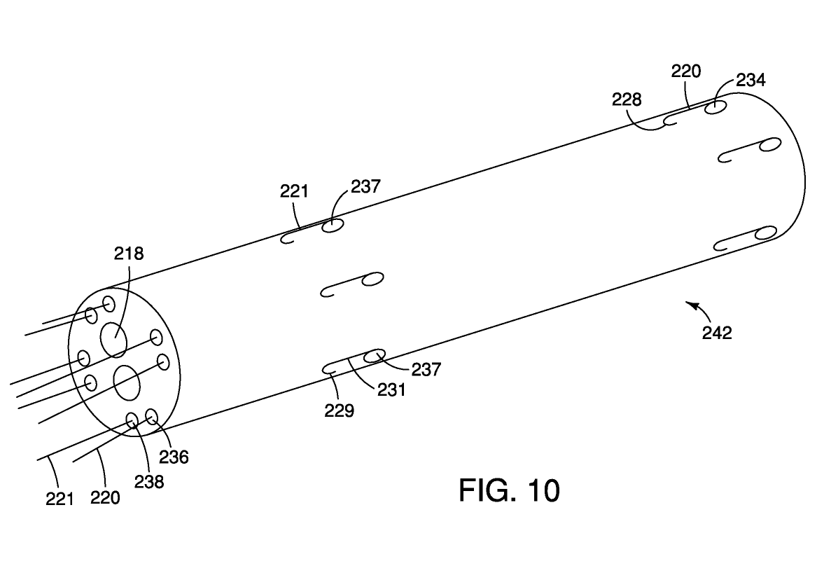

FIG. 9 illustrates a perspective view of a distal portion 212 of a steerable catheter 200 according to an embodiment of the invention. The catheter 200 includes an elongate shaft 214 extending from the distal portion 212 to a proximal portion 216. The elongate shaft 214 includes one or more lumens 218 extending at least partially therethrough. The steerable catheter 200 further includes a first plurality of control cables 220 extending through at least a portion of the elongate shaft 214. One or more of the first plurality of control cables 220 extend outward from an outer surface 224 of the shaft 214 at the distal portion 212 in a first position 226 as shown in FIG. 9. The first plurality of control cables 220 may also be retracted to a second position 242 as shown in FIG. 10. A distal end 228 of each control cable 220 may be secured relative to the shaft 214 with a remaining portion 232 of the control cable 220 movably positionable relative to the shaft 214. The distal portion 212 of the shaft 214 may include a plurality of openings 234 in a wall 225 through which the plurality of control cables 220 may movably extend. As shown in FIG. 9, the openings 234 may be positioned proximal to a distal end 235 of the shaft 214. The plurality of control cables 220 may extend through a plurality of cable lumens 236 to the plurality of openings 234 and out through the openings 234. Each cable 220 may have its own lumen 236 and opening 234. The control cables 220 may be connected to a handle (not shown) similar to the handles described above. The first plurality of control cables 220 may also be positioned in recesses formed in the wall 225 in the second position 242 similar to the positioning of the control cables 20 in the recesses discussed above with reference to FIGS. 2 and 3.

As shown in FIG. 9, the steerable catheter 200 may also include a second plurality of control cables 221 extending through at least a portion of the elongate shaft 214. One or more of the plurality of control cables 221 extend outward from the outer surface 224 of the shaft 214 at the distal portion 212 and are positioned proximal to the first plurality of control cables 220 in a first position 226 as shown in FIG. 9. The second plurality of control cables 221 may also be retracted to a second position 242 as shown in FIG. 10. The first and second pluralities of control cables 220, 221 may also be moved to the first position 226 or the second position 242 independent of each other so that one for the first and second pluralities of control cables 220, 221 is in the first position 226 and the other of first and second pluralities of control cables 220, 221 is in the second position 242. A distal end 229 of each of the second plurality of control cables 221 may be secured relative to the shaft 214 with a remaining portion 233 of the control cable 221 movably positionable relative to the shaft 214. The distal portion 212 of the shaft 214 may include a plurality of openings 237 in the wall 225 through which the second plurality of control cables 221 may movably extend. As shown in FIG. 9, the openings 237 may be positioned proximal to the openings 234 of the shaft 214. The second plurality of control cables 221 may extend through a plurality of cable lumens 238 to the plurality of openings 234 and out through the openings 237. Each cable 221 may have its own lumen 238 and opening 237. The second plurality of control cables 221 may be connected to a handle (not shown) similar to the handles described above. The combination of the first and second plurality of control cables 220, 221 allows for additional directions of movement of the distal portion 212 of the catheter 200 as will be understood by one skilled in the art. In some embodiments, the distal portion 212 may also include a hinge or a pivot 228 to allow for additional flexibility and movement of the distal end portion 227. As shown in FIG. 9, the pivot 228 may be positioned between the first and second plurality of control cables 220, 221.

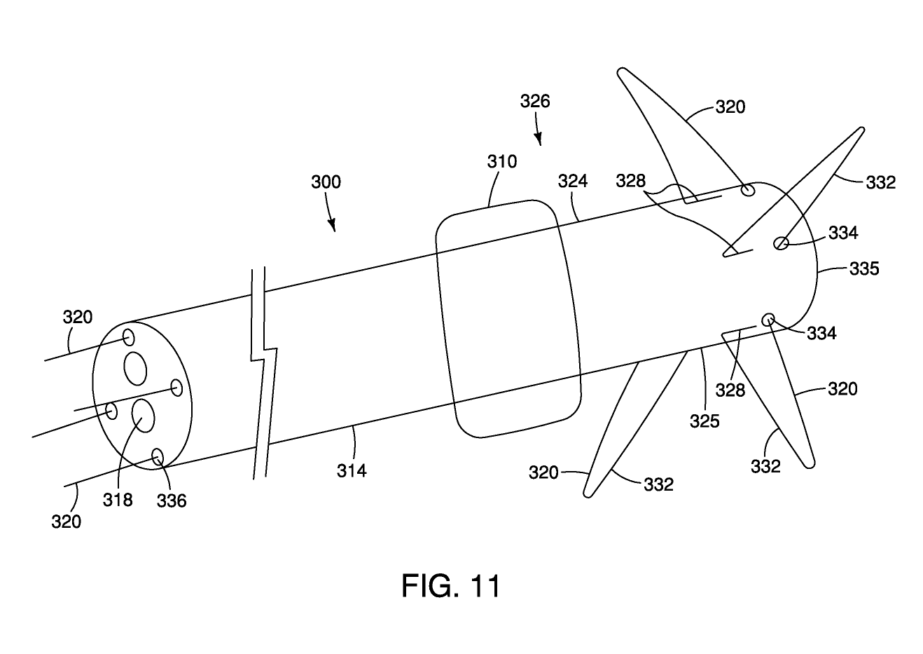

In some embodiments, a steerable catheter 300 may also include a balloon 310. The balloon 310 may be used to secure the catheter 300 in a longitudinal position within the patient's lumen and allow a distal portion 312 of the catheter 300 to be moved into proper position for treatment using one or more control cables 320. The balloon 310 is shown in FIG. 11 and may be provided to circumferentially surround the catheter 300. The balloon 310 may also be used with steerable catheters having the first and second plurality of control cables as described above with reference to FIGS. 9 and 10. The catheter 300 includes an elongate shaft 314. The elongate shaft 314 includes one or more lumens 318 extending at least partially therethrough. One of the lumens 318 may be an inflation lumen, operably connected to the balloon 310 for inflation and deflation of the balloon 310. The balloon 310 is deflatable for delivery and movement of the catheter 100 within a patient's lumen. The steerable catheter 300 further includes a first plurality of control cables 320 extending through at least a portion of the elongate shaft 314. One or more of the first plurality of control cables 320 extend outward from an outer surface 324 of the shaft 314 at the distal portion 312 in a first position 326 as shown in FIG. 11 and have a curvilinear shape. The first plurality of control cables 320 may also be retracted to a second position similar to the retracted positions described above. A distal end 328 of each control cable 320 may be secured relative to the shaft 314 with a remaining portion 332 of the control cable 220 movably positionable relative to the shaft 314. The distal portion 312 of the shaft 214 may include a plurality of openings 334 in a wall 325 through which the plurality of control cables 320 may movably extend. The plurality of control cables 320 may extend through a plurality of cable lumens 336 to the plurality of openings 334 and out through the openings 334. Each cable 320 may have its own lumen 336 and opening 334. The opening 334 may be spaced apart from where the distal end 328 is secured to the shaft 314 to facilitate the cable 320 forming a curvilinear shape extending away from the shaft 314. The control cables 320 may be connected to a handle (not shown) similar to the handles described above.

The materials used to manufacture the components of the steerable catheter described herein may be any materials known to one skilled in the art that are suitable for use in patients. By way of non-limiting example, the shaft may be formed from polytetrafluoroethylene (PTFE) particularly when a low friction shaft is desirable. Nylon and HDPE may also be used for clarity. Additional possible materials include, but are not limited to the following, polyethylene ether ketone (PEEK), fluorinated ethylene propylene (FEP), perfluoroalkoxy polymer resin (PFA), polyamide, polyurethane, high density or low density polyethylene, and nylon including multi-layer or single layer structures and the like and may also include reinforcement wires, braid wires, coils, coil springs and or filaments. In some embodiments, the diameter of the shaft will depend on the type of lumen in which the catheter is being inserted. By way of non-limiting example, the steerable catheter may be about 1-6 mm for insertion into the biliary tract. The shaft of a catheter for insertion through a 4.2 mm accessory channel of a duodenoscope and into the bile duct may have a diameter of about 1-3.5 mm and the shaft of a sphincterotome, about 1-3 mm. The diameter of the shaft of the catheter may also be larger, for example, for catheters to be inserted into the esophagus. The steerable catheters described herein may also be used in the upper and lower gastrointestinal tracts.

The control cables may be made from any material suitable for movably positioning the distal portion of the shaft of the catheter and flexible enough to limit the damage to the lumen from the cables. The control cables may be monofilament, braided, twisted or multifilament. In some embodiments, the control cables may be flattened, ribbon-shaped cables. The cables may be formed from polymers or a metallic alloy such as stainless steel or nickel titanium or any suitable material. In some embodiments, the control cables may be about 0.005 to about 0.035 inches thick (about 0.127 mm to about 0.9 mm).

Other suitable biocompatible materials may also be used for any of the components described herein.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. A steerable catheter comprising:
a flexible elongate shaft having a proximal end portion, a distal end portion and a cable lumen extending from the proximal end portion to the distal end portion, the cable lumen having an opening at the distal end portion;
a control cable having a first portion and a second portion, the first portion of the control cable fixed in position relative to the shaft and the second portion movable relative to the shaft, at least a portion of the second portion extendable through at least a portion of the cable lumen and extendable through the opening, the control cable having a first position wherein the at least portion of the second portion is axially extendable away from a wall of the shaft and a second position for delivery of the catheter; and
a handle operably connected to the control cable for moving the control cable from the second position to the first position.

2. The steerable catheter of claim 1, further comprising a plurality of control cables.

3. The steerable catheter of claim 2, further comprising a plurality of cable lumens and openings, each of the plurality of control cables extending at least partially through one of the plurality of cable lumens.

4. The steerable catheter of claim 2, wherein each of the plurality of control cables is independently movable.

5. The steerable catheter of claim 3, wherein the openings are equally spaced around a circumference of the shaft.

6. The steerable catheter of claim 1, wherein the handle comprises an actuator operably connected to the control cable to move the control cable from the second position to the first position.

7. The steerable catheter of claim 6, wherein the actuator is distally movable along a longitudinal axis of the shaft to move the control cable to the first position so that the at least the portion of the second portion is axially extendable.

8. The steerable catheter of claim 6, wherein the actuator is rotatably movable to change the position of the control cable.

9. The steerable catheter of claim 2, wherein the catheter further comprises a second plurality of control cables and a second plurality of openings, a portion of each of the second plurality of control cables axially extendable through one of the second plurality of openings, the second plurality of openings positioned proximal to the first plurality of openings.

10. The steerable catheter of claim 1, wherein the shaft further comprises a pivot member positioned proximal to the opening.

11. The steerable catheter of claim 1, wherein the catheter further includes a balloon positioned proximal to the opening.

12. A steerable catheter comprising:
a flexible elongate shaft having a proximal end portion, a distal end portion and a plurality of cable lumens extending from the proximal end portion to the distal end portion, each of the plurality of cable lumens having an opening at the distal end portion;
a plurality of control cables, each of the plurality of control cables having a first portion and a second portion, the first portion fixed in position relative to the shaft and the second portion movable relative to the shaft, each of the second portions having at least a portion thereof extendable through one the plurality of cable lumens and out through the opening and axially away from a wall of the shaft in a first position; and
a handle operably connected to the plurality of control cables to move one or more of the control cables from a second position to the first position.

13. The steerable catheter of claim 12, wherein the handle comprises an actuator, the actuator operable to move one or more of the plurality of control cables from the second position to the first position.

14. The steerable catheter of claim 13, wherein the actuator is distally movable along a longitudinal axis of the shaft to move one or more of the plurality of control cables to the first position.

15. The steerable catheter of claim 12, wherein each of the control cables has a curvilinear shape in the first position.

16. The steerable catheter of claim 12, wherein the distal end portion is moveable in at least four different directions by moving the plurality of control cables relative to each other.

17. A method of steering a catheter, the method comprising:
moving a second portion of a first control cable from a second cable position to a first cable position and a first portion of the first control cable remains fixed in position in relation to a shaft of the catheter;
extending at least a portion of the second portion through an opening in the shaft and axially away from a wall of the shaft; and
contacting a surface with the portion of the second portion so that a distal portion of the shaft is moved from a first shaft position to a second shaft position.

18. The method of claim 17, further comprising moving a second portion of a second control cable from a second cable position to a first cable position and a first portion of the second control cable remains fixed in position in relation to the shaft of the catheter and moving the shaft from the second shaft position to a third shaft position or to the first shaft position.

19. The method of claim 17, further comprising distally moving an actuator along a longitudinal axis of the shaft to move the first control cable to the first cable position.

20. The method of claim 18, comprising moving the first and second control cables independent of each other.
